(19) United States
(12) Patent Application Publication
(10) Pub. No.: US 2010/0234314 A1
(43) Pub. Date: Sep. 16, 2010

(54) IDARUBICIN FOR THE TREATMENT OF LYMPHOMA IN A DOG

(75) Inventors: Pamela Jo Berlinski, Parchment, MI (US); Steven Glenn Kamerling, Kalamazoo, MI (US)

Correspondence Address: PHARMACIA & UPJOHN, 7000 Portage Road, KZO-300-104, KALAMAZOO, MI, 49001, US

(73) Assignee: PFIZER INC., New York, NY (US)

(21) Appl. No.: 12/682,226

(22) PCT Filed: Oct. 15, 2008
(86) PCT No.: PCT/IB08/02783

§ 371 Date: May 11, 2010

Related U.S. Application Data
(60) Provisional application No. 60/982,843, filed on Oct. 26, 2007.

(51) Int. Cl. A61K 31/704; C07H 15/252; A61P 35/00

(52) U.S. Cl. 514/34; 536/6.4

(57) ABSTRACT

The present invention relates to a method of treating a lymphoma in a dog comprising administering to a dog in need of such treatment a therapeutically effective amount of idarubicin or a pharmaceutically acceptable salt thereof.

The present invention relates to the use of idarubicin for the treatment of canine cancer. In particular it relates to the use of idarubicin for the treatment of canine lymphoma.

### BACKGROUND OF THE INVENTION

Between 10 and 25% of dogs will develop cancer in their lifetime, and dogs living for 10 years or more have a 50% chance of developing cancer. Even so, there are currently no drugs approved for the treatment of canine cancers.

The most prevalent cancer in dogs is lymphoma. Chemotherapy options for canine lymphoma include off-label use of the anthracycline agent doxorubicin. However, the use of doxorubicin is generally restricted to veterinary oncologists because it must be administered by slow intravenous infusion. The administration is associated with a risk of severe tissue toxicity if extravasation occurs, and shock if the patient develops an allergic reaction. The dose-limiting side effect of doxorubicin is generally neutropenia. The chronic use of doxorubicin is further limited by its propensity to cause cardiac toxicity, and by the development of drug resistance.

There is therefore a continuing need for further chemotherapy options for the treatment of canine lymphomas. Preferably, such options would allow for a more convenient route of administration, such that the administration could be carried out by a non-specialist. Therapies that are less prone to the development of resistance would be particularly valuable, as would therapies that could be used in animals that have already developed resistance to doxorubicin. Preferred therapeutic options would be those that do not have adverse interactions with other chemotherapeutic agents, and particularly preferred options would be agents that provide synergistic results when used in combination with other agents.

Idarubicin (I) is an anthracycline agent which has been approved for use in human chemotherapy, and which can be administered to human patients by both the intravenous and oral routes. Idarubicin is available for injection both generically and under the name Idamycin®. An oral formulation is marketed in Europe under the name Zavedos. It is metabolised, especially following oral administration, to idarubicinol (II), which is also an effective cytotoxic agent.

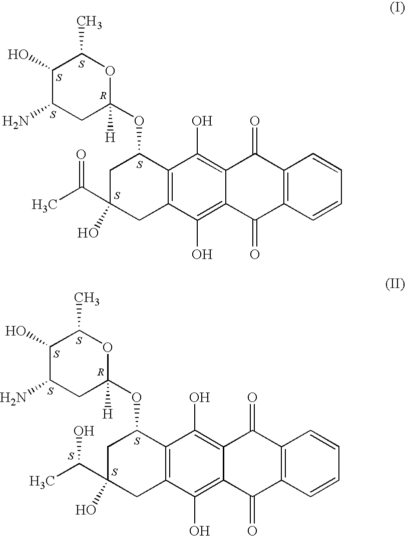

The effects of idarubicin in cats have been briefly investigated (Moore et al., J. Am. Vet. Med. Assoc. 1995, 206(10), 1550-1554). There are no reports of the therapeutic use or pharmacokinetics of idarubicin in dogs. We have now found that idarubicin is particularly suitable for the treatment of canine lymphomas. It may conveniently be administered orally, and oral bioavailability is not adversely affected by feeding. It is also active against tumours that have developed resistance to doxorubicin.

### SUMMARY OF THE INVENTION

In a first aspect, the present invention provides a method of treating a lymphoma in a dog comprising administering to a dog in need of such treatment a therapeutically effective amount of idarubicin or a pharmaceutically acceptable salt thereof.

In another aspect, the present invention provides the use of idarubicin or a pharmaceutically acceptable salt thereof for the manufacture of a medicament for treating a lymphoma in a dog.

In a further aspect, the present invention provides a pharmaceutical composition for treating a lymphoma in a dog comprising idarubicin or a pharmaceutically acceptable salt thereof and a pharmaceutically acceptable carrier.

### DETAILED DESCRIPTION OF THE INVENTION

In general, the invention concerns the use of idarubicin (I) for the treatment of lymphomas in dogs. The use of idarubicin allows for a treatment regime that may be equal or superior to currently used protocols. The possibility of oral administration may be more convenient and provide for improved safety.

As used herein:
the term “treatment” includes palliative (inhibition of disease progression), curative (induction of remission) and prophylactic (maintenance of remission), and rescue treatment in the case of disease relapse;
the term “dog” includes all breeds and varieties of domestic dog, as well as non-domesticated species (such as wolves and foxes) that are in zoological collections or are part of a captive breeding program.

The idarubicin may be used in its free-base form or in the form of a pharmaceutically acceptable salt. As used herein, “pharmaceutically acceptable” includes “veterinarily acceptable”. Pharmaceutically acceptable salts of idarubicin include the acid addition salts thereof.

Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

A particularly preferred salt is idarubicin hydrochloride.

When the idarubicin is administered in solid form then the use of any suitable solid form is foreseen. Thus the present invention is not limited to any particular solvated or unsolvated form, nor to any particular polymorphic form.

Idarubicin suitable for the purposes of the present invention may be obtained from Tecoland Corporation, Nerviano Medical Sciences (Italy), Pfizer Cork (Ireland) or TPM Antibioticos S.p.A (Italy). Alternatively Idarubicin may be prepared according to the methods disclosed in EP337665 and references therein. Idarubicin hydrochloride suitable for the purposes of the present invention may be obtained from Transo-pharm.

Idarubicin for use in the invention will generally be formulated in a manner appropriate to the desired route of administration to the subject dog. The formulation may comprise one or more pharmaceutically acceptable excipients, such as are well known in the art.

In a preferred embodiment, the idarubicin is administered orally. Formulations suitable for oral administration include solid and liquid formulations.

Solid formulations include tablets, flavoured tablets, capsules containing particulates, liquids, or powders; lozenges (including liquid-filled), chews; multi- and nano-particulates; gels, solid solution, liposome, films (including muco-adhesive), ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colorants, flavouring agents, preservatives and taste-masking agents.

Capsules may be made from, for example, hard or soft gelatin. The gelatin may be mixed with, for example, a dyestuff (such as red iron oxide) or an opacifier (such as titanium dioxide). The capsule may be filled with, for example, a powder comprising the active agent and excipients such as disintegrants, lubricants and structural matrices.

An example of a suitable filling for a capsule formulation is a powder which is made up of idarubicin hydrochloride (5 wt %), microcrystalline cellulose (93 wt %) and glyceryl palmito-stearate (2 wt %). 1 kg of this mixture is sufficient for 10,000 capsules each containing 5 mg of idarubicin hydrochloride.

In an alternative embodiment, the idarubicin is administered parenterally, i.e. directly into the blood stream, directly into a tumour (intratumour), or into an internal organ. Suitable means for parenteral administration include intravenous, intraarterial, and intravesicular.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

Idarubicin may be used according to the present invention to treat canine lymphomas.

The dose of idarubicin to be administered will be determined by the veterinarian, taking into account the size of the dog, the progression of the disease, and any other relevant factors. Typically, the dose of idarubicin may be between 0.4 and 1.0 mg/kg when given orally to dogs weighing less than 15 kg and between 9 and 25 mg/meter squared to dogs weighing greater than or equal to 15 kg. Unit dosage forms (tablets or capsules) providing doses of 0.5 mg, 1 mg, 5 mg, 10 mg and 25 mg allow for the convenient treatment of most breeds of dog.

The dose may be repeated at suitable intervals, such as for example once a week, once every two weeks, once every three weeks, or once a month, until the desired outcome is achieved. It is possible that idarubicin will be dosed metronomically, in which small daily doses are given to induce an antiangiogenic effect or limit disease progression.

For intravenous administration the dose will be lower, such as for example between 0.03 and 3 mg/kg, 0.05 and 1.5 mg/kg, or 0.1 and 1 mg/kg.

### Example 1

Study to determine the maximum tolerated oral dose (MTD) and dose limiting toxicities (DLT) of PF-00929868-01 (idarubicin) in client-owned dogs (weighing greater than or equal to 15 kg) with lymphoma.

Three dogs suffering from lymphoma and weighing greater than or equal to 15 kg were treated with a single treatment of oral idarubicin at a dose of 12.5 mg/m². This dose was well tolerated for a period of 3 weeks.

### Example 2

### In Vitro Assessment of Idarubicin on Lymphoproliferation in Canine Lymphoma Biopsies and Lymphoma Cell Lines

Individual IC₅₀'s anti-proliferative values in response to idarubicin and doxorubicin were compared. The study utilized an in vitro assay run with ex-vivo derived canine lymphoma cells and with established cell lines maintained over many passages using standard tissue culture techniques. Nodal tissues were obtained from canine lymphoma patients at the MSU Veterinary Clinic. Assays were run within 24 hours of receipt nodal tissues or before the 10th passage from frozen cell line stocks.

The established cell lines used were 3132 and Cl-1 which are canine lymphoma cell lines of B-cell and T-cell origin, respectively. These cells were cultured in RPMI complete media in a humidified incubator with 5% CO₂.

### Materials and Methods:

Anti-proliferation assay: (1) Canine Lymphoma Cell Lines. The 3132 and Cl-1 are canine lymphoma cell lines of B-cell and T-cell origin, respectively. These cells were cultured in RPMI complete media (Advanced RPMI 1640, 10 mM Hepes, 2 mM Glutamax, 100 U/mL penicillin, 100 ug/mL streptomycin and 0.25 ug/mL Amphotercin B) supplemented with either 10% FBS (3132) or 20% FBS (CL-1) at 37° C. in a humidified incubator with 5% CO₂. (2) Ex vivo canine lymphoma nodal tissue. Malignant lymph nodes were excised by veterinary staff at Michigan State University (MSU) Veterinary College, placed into transport media (Advanced RPMI 1640 complete medium supplemented with 10% Fetal Bovine Serum (FBS), 100 U/mL penicillin, 100 ug/mL streptomycin and 0.25 ug/mL Amphotercin B (Invitrogen/Gibco®). Nodes were processed within 24 hours of removal by mincing into tiny pieces and passing through a tissue sieve. Cell suspensions were spun at 200×g, supernatant was removed, and the cell pellet was resuspended in NH₄Cl (0.15M) for 10 minutes at room temperature. The cell suspension was pelleted by centrifugation; the NH₄Cl was removed and washed once with Hanks Balanced Salt Solution (HBSS), followed by re-suspension in Proliferation Medium (Advanced RPMI complete, 1% FBS, 50 nM 2-Mercaptoethanol, 100 U/mL penicillin, 100 ug/mL streptomycin and 0.25 ug/mL Amphotercin B). The cell suspension was then passed through a 100 μm nylon cell strainer (BD-Falcon) and counted using a hemacytometer. Cells were cultured in either Proliferation Medium alone, Proliferation Medium supplemented with 0.005% Pansorbin® (Heat inactivated, formalin-fixed Staphylococcus Aureus cells (SAC), Calbiochem), and 10 ng/mL canine IL-2 (R&D Systems), or Proliferation Medium supplemented with 125 ng/mL concavalin A (Sigma) and 125 ng/mL lipopolysaccamide (LPS; Calbiochem). (3) In vitro Anti-Proliferation Assay Method. Cells cultured in medium described above were plated in 96-well Costar plates (Corning) at a density of 1×103 cells/well (lymphoma cell lines) or 2×105 cells/well (lymph node cells) and exposed to various concentrations of test compounds for up to 5 days at 37° C. in a humidified incubator with 5% CO₂. Effects on proliferation were determined using the CellTiter 96® Aqueous Non-Radioactive Cell Proliferation Assay (Promega) according to manufacturer's instructions. In general, proliferation was indirectly measured using a soluble tetrazolium salt (MTS) and an electron coupling agent. MTS bioreduction into a formazan product soluble in tissue culture medium was monitored by absorbance at 490 nM on a Spectramax plate reader using Softmax Pro 4.6 software (Molecular Devices). Data were graphically displayed as percent control using GraphPad Prism 4.00, and IC₅₀ curves were fitted using a non-linear regression model with a sigmoidal dose response. (4) Data Analysis: For nodal tissue, data were handled in the following manner through Prism 4.0 (Graphpad Software). Mean values from raw optical density values (OD) were calculated for the Un-stimulated, Simulated and all Drug Treatment (triplicates) and used in the following formula:

(Mean Drug Treatment OD−Mean Un-stim OD)×100=% Control Proliferation

(Mean Stim OD−Mean Un-stim OD)

For cell lines:

(Mean Drug Treatment OD−Mean media blank OD)×100=% Control Proliferation

(Mean Untreated OD−Mean media blank OD)

Once calculated, these values for each drug concentration (or standard) were graphed and an IC₅₀ calculated by a point to point analysis. Percent control values were not allowed to exceed 100% or fall below 0% for analysis of IC₅₀ values. IC₅₀ values for each lymph node and cell line are reported here.

### Doxorubicin and Idarubicin Anti-Proliferation IC₅₀'s

| | | Stimulant in | | |
|---|---|---|---|---|
| Cell Line or | | Culture | Doxorubicin | Ioxorubicin |
| Lymph Node | Description | Medium | IC₅₀ (nM) | IC₅₀ (nM) |
| 3132 | B-cell LSA line | | | 2.7 |
| CL-1 | T-cell LSA line | | 4.3 | 1.9 |
| MSU LN 1 | Chemotherapy | LPS + ConA | 55 | 7.5 |
| ID618801 | Resistant | | | |
| | B-cell LSA | | | |
| MSU LN 1 | | SAC + IL-2 | 11 | 7.5 |
| MSU LN 2 | De novo | | | |
| ID401776 | B-cell LSA | | | |
| MSU LN 6 | Chemotherapy | LPS + ConA | 28 | 3 |
| ID401557 | Resistant | | | |
| | T-cell LSA | | | |
| MSU LN 6 | | SAC + IL-2 | 13 | 2.5 |
| MSU LN 7 | Chemotherapy | LPS + ConA | 10 | 2.5 |
| ID663780 | Resistant | | | |
| | T-cell LSA | | | |
| MSU LN 8 | De novo T-cell | LPS + ConA | 9 | 4 |
| ID668165 | lymphoma | | | |
| MSU LN 8 | | SAC + IL-2 | 7 | 3 |
| MSU LN 10 | Chemotherapy | LPS + ConA | >100 | 56 |
| ID401979 | Resistant | | | |
| | B-cell LSA | | | |
| MSU LN 11 | De novo | LPS + ConA | ND | 4 |
| ID671795 | B-cell LSA | | | |
| MSU LN 14 | De novo | Con A | ND | 11 |
| ID403527 | T-cell LSA | | | |

*ND = Not done
LPS = Lipopolysaccharide
ConA = Concanavalin A
SAC = Staphylococcus Aureus Cowan
IL 2 = Interleukin 2

Idarubicin (IDA) and doxorubicin produced dose dependent inhibition of the proliferation of canine lymphoma nodal tissues. They also inhibited the proliferation of canine lymphoma cell lines. Idarubicin was more potent and cytotoxic than doxorubicin against all nodal and cell line lymphomas regardless of B or T-cell lineage. Idarubicin was more effective in inhibiting proliferation of all nodal tissue obtained from dogs diagnosed with chemotherapy resistant lymphomas doxorubicin.

Although the invention has been described above with reference to the disclosed embodiments, those skilled in the art will readily appreciate that the specific experiments detailed are only illustrative of the invention. It should be understood that various modifications can be made without departing from the spirit of the invention. Accordingly, the invention is limited only by the following claims.

1. A method of treating a lymphoma in a dog comprising administering to a dog in need of such treatment a therapeutically effective amount of idarubicin or a pharmaceutically acceptable salt thereof.

2. The method of claim 1 wherein the idarubicin is administered orally.

3. The method of claim 2 wherein the idarubicin is administered as a pharmaceutically acceptable salt.

4. The method of claim 3 wherein the idarubicin is administered as the hydrochloride salt.

5. The method of claim 1 wherein the lymphoma is a doxorubicin-resistant lymphoma.

6. The method of claim 1 wherein the dog is presenting doxorubicin-induced cardio-toxicity.

7. The use of idarubicin or a pharmaceutically acceptable salt thereof for the manufacture of a medicament for treating a lymphoma in a dog.

8. The use of claim 7 wherein the idarubicin is to be administered orally.

9. The use of claim 8 wherein the idarubicin is to be administered as a pharmaceutically acceptable salt.

10. The use of claim 9 wherein the idarubicin is to be administered as the hydrochloride salt.

11. A pharmaceutical composition for treating a lymphoma in a dog comprising idarubicin or a pharmaceutically acceptable salt thereof and a pharmaceutically acceptable carrier.

12. The composition of claim 11 which is adapted for oral administration.

13. The composition of claim 12 wherein the idarubicin is present as a pharmaceutically acceptable salt.

14. The composition of claim 13 wherein the idarubicin is present as the hydrochloride salt.
